(19) United States
(12) Patent Application Publication
(10) Pub. No.: US 2025/0367424 A1
(43) Pub. Date: Dec. 4, 2025

(54) CATHETER CONNECTION MANIFOLD BYPASS ACCESSORY DEVICE

(71) Applicant: Boston Scientific Scimed, Inc., Maple Grove, MN (US)

(72) Inventors: Carl Parent, Brooklyn Park, MN (US); David Raab, Roseville, MN (US); Robert M. Wold, Maple Lake, MN (US); Joel N. Groff, Delano, MN (US); Scott McGhee, Ottawa (CA); Patrick A. Haverkost, Crystal, MN (US)

(21) Appl. No.: 19/223,848

(22) Filed: May 30, 2025

Related U.S. Application Data
(60) Provisional application No. 63/654,422, filed on May 31, 2024.

(51) Int. Cl. A61M 39/12; A61M 39/00; A61M 39/10

(52) U.S. Cl. CPC A61M 39/12; A61M 2039/0009; A61M 2039/1038; A61M 2205/0216

(57) ABSTRACT

Embodiments herein relate to catheter connection manifold bypass devices, cancer therapy delivery systems, and related methods. For example, a catheter connection manifold bypass device can be included having a fluid passage conduit with a proximal end and a distal end. The bypass device can also include a deformable tip disposed around the distal end of the fluid passage conduit. The deformable tip can fit within a distal portion of a cavity within a connection manifold for a fluid delivery catheter. The bypass device can include a proximal connection port that can be connected to the proximal end of the fluid passage conduit. The bypass device can also include a connection adapter defining a channel, wherein the fluid passage conduit passes through the channel. The distal end of the connection adapter can be configured to attach to a proximal end of the fluid delivery catheter. Other embodiments are also included herein.

This application claims the benefit of U.S. Provisional Application No. 63/654,422, filed May 31, 2024, the content of which is herein incorporated by reference in its entirety.

### FIELD

Embodiments herein relate to catheter connection manifold bypass devices, cancer therapy delivery systems, and related methods.

### BACKGROUND

According to the American Cancer Society, cancer accounts for nearly 25% of the deaths that occur in the United States each year. Cancerous tumors can form if one normal cell in any part of the body mutates and then begins to grow and multiply rapidly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Chemotherapy and immunotherapy are other common therapeutic approaches but can include substantial side effects. The use of radiation represents another approach. Specifically, radiation therapy aims at damaging the DNA of cancer cells so that they lose the capability to divide and proliferate, thus leading to the cell death process for the cancerous cells.

Brachytherapy is a form of radiation therapy where a sealed radiation source is placed inside or next to the area requiring treatment. As one form of brachytherapy, targeted radioembolization therapy can be used to treat unresectable tumors. For example, Y-90 glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy.

### SUMMARY

Embodiments herein relate to catheter connection manifold bypass devices, cancer therapy delivery systems, and related methods. In a first aspect, a catheter connection manifold bypass device can be included having a fluid passage conduit. The fluid passage conduit can include a proximal end and a distal end. The bypass device can further include a deformable tip, wherein the deformable tip can be disposed around the distal end of the fluid passage conduit and wherein the deformable tip can be configured to fit within a distal portion of a cavity within a connection manifold for a fluid delivery catheter. The bypass device can further include a proximal connection port, wherein the proximal connection port can be connected to the proximal end of the fluid passage conduit. The bypass device can further include a connection adapter, the connection adapter defining a channel, wherein the fluid passage conduit passes through the channel. A distal end of the connection adapter can be configured to attach to a proximal end of the fluid delivery catheter.
In a second aspect, in addition to one or more of the preceding or following aspects, or in the alternative to some aspects, the bypass device reduces and/or eliminates step-changes in an inner diameter of a fluid passage way passing through the bypass device and the fluid delivery catheter when the two are connected.
In a third aspect, in addition to one or more of the preceding or following aspects, or in the alternative to some aspects, the fluid passage conduit can be configured to be advanced into the fluid delivery catheter such that a distal end of the fluid passage conduit comes to rest at a distal end of the cavity within a connection manifold at the proximal end of the fluid delivery catheter.
In a fourth aspect, in addition to one or more of the preceding or following aspects, or in the alternative to some aspects, the proximal connection port can be configured to engage with an outflow conduit of a cancer therapy suspension forming assembly.
In a fifth aspect, in addition to one or more of the preceding or following aspects, or in the alternative to some aspects, the proximal connection port can be physically integrated with an outflow conduit of a cancer therapy suspension forming assembly.
In a sixth aspect, in addition to one or more of the preceding or following aspects, or in the alternative to some aspects, the fluid passage conduit can be rigid.
In a seventh aspect, in addition to one or more of the preceding or following aspects, or in the alternative to some aspects, the fluid passage conduit can be formed of a metal or a polymer.
In an eighth aspect, in addition to one or more of the preceding or following aspects, or in the alternative to some aspects, the deformable tip can be formed of a material different than the fluid passage conduit.
In a ninth aspect, in addition to one or more of the preceding or following aspects, or in the alternative to some aspects, the deformable tip can be formed of an elastomeric material.
In a tenth aspect, in addition to one or more of the preceding or following aspects, or in the alternative to some aspects, the deformable tip includes a tapered distal end.
In an eleventh aspect, in addition to one or more of the preceding or following aspects, or in the alternative to some aspects, the proximal connection port can include a luer connector.
In a twelfth aspect, in addition to one or more of the preceding or following aspects, or in the alternative to some aspects, the connection adapter can be a Tuohy Borst adapter.
In a thirteenth aspect, in addition to one or more of the preceding or following aspects, or in the alternative to some aspects, the fluid delivery catheter can have a fluid delivery channel with an inner diameter of less than 0.040 inches.
In a fourteenth aspect, in addition to one or more of the preceding or following aspects, or in the alternative to some aspects, the fluid delivery catheter can be a neurocatheter.
In a fifteenth aspect, a catheter connection manifold bypass device can be included having a fluid passage conduit. The fluid passage conduit can include a proximal end and a distal end. The bypass device can include a deformable tip, wherein the deformable tip can be disposed around the distal end of the fluid passage conduit. The deformable tip can be configured to fit within a distal portion of a cavity within a connection manifold for a fluid delivery catheter. The bypass device can also include a connection housing. The connection housing can include a proximal connection fitting, wherein the proximal connection fitting can be configured to engage with an outflow conduit of a cancer therapy suspension forming assembly. The fluid passage conduit can pass through the connection housing. The bypass device can further include a rotating connector element, the rotating connector element defining a channel, wherein the fluid passage conduit passes through the channel. A distal end of the rotating connector element can be configured to attach to a proximal end of the fluid delivery catheter and the rotating connector element engages with the connection housing.
In a sixteenth aspect, in addition to one or more of the preceding or following aspects, or in the alternative to some aspects, the bypass device reduces and/or eliminates step-changes in an inner diameter of a fluid passage way passing through the bypass device and the fluid delivery catheter when the two are connected.
In a seventeenth aspect, in addition to one or more of the preceding or following aspects, or in the alternative to some aspects, wherein rotation of the rotating connector element causes the fluid passage conduit to be advanced into the fluid delivery catheter.
In an eighteenth aspect, in addition to one or more of the preceding or following aspects, or in the alternative to some aspects, the fluid passage conduit can be configured to be advanced into the fluid delivery catheter such that a distal end of the fluid passage conduit comes to rest at a distal end of the cavity within a connection manifold at the proximal end of the fluid delivery catheter.
In a nineteenth aspect, in addition to one or more of the preceding or following aspects, or in the alternative to some aspects, the proximal connection fitting can be physically integrated with the outflow conduit of the cancer therapy suspension forming assembly.
In a twentieth aspect, a cancer therapy delivery system can be included having a suspension generation assembly and a catheter connection manifold bypass device. The catheter connection manifold bypass device can be in fluid communication with the suspension generation assembly. The catheter connection manifold bypass device can include a fluid passage conduit, the fluid passage conduit can include a proximal end, and a distal end. The bypass device can include a deformable tip, wherein the deformable tip can be disposed around the distal end of the fluid passage conduit. The deformable tip can be configured to fit within a distal portion of a cavity within a connection manifold for a fluid delivery catheter. The bypass device can include a proximal connection port, wherein the proximal connection port can be connected to the proximal end of the fluid passage conduit. The bypass device can include a connection adapter, the connection adapter defining a channel, wherein the fluid passage conduit passes through the channel. A distal end of the connection adapter can be configured to attach to a proximal end of the fluid delivery catheter.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cancer therapy suspension forming assembly in accordance with various embodiments herein.

FIG. 2 is a schematic view of an outflow line of a suspension forming assembly and a fluid delivery catheter in accordance with various embodiments herein.

FIG. 3 is a schematic view of a fluid delivery catheter in accordance with various embodiments herein.

FIG. 4 is a schematic view of a catheter connection manifold bypass device in accordance with various embodiments herein.

FIG. 5 is a schematic view of a catheter connection manifold bypass device connecting with a fluid delivery catheter in accordance with various embodiments herein.

FIG. 6 is a schematic view of suspension forming assembly and a bypass device connecting with a fluid delivery catheter in accordance with various embodiments herein.

FIG. 7 is a schematic view of a needle manifold insert in accordance with various embodiments herein.

FIG. 8 is a schematic view of alternative embodiments of a fluid passage conduit and deformable tip thereof in accordance with various embodiments herein.

FIG. 9 is a schematic view of a distal portion of the outflow line of a suspension forming assembly in accordance with various embodiments herein.

FIG. 10 is a schematic view of components of a connection manifold bypass device in accordance with various embodiments herein.

FIG. 11 is a schematic view of components of a connection manifold bypass device in accordance with various embodiments herein.

FIG. 12 is a schematic view of a bypass device integrated with the outflow line of a suspension forming assembly in accordance with various embodiments herein.

FIG. 13 is a schematic view of a distal tip of a fluid passage conduit in accordance with various embodiments herein.

FIG. 14 is a schematic view of a catheter connection manifold bypass device in accordance with various embodiments herein.

FIG. 15 is a schematic view of a catheter connection manifold bypass device in accordance with various embodiments herein.

FIG. 16 is a schematic view of a catheter connection manifold bypass device in accordance with various embodiments herein.

FIG. 17 is a schematic view of a catheter connection manifold bypass device in accordance with various embodiments herein.

FIG. 18 is a schematic view of a catheter connection manifold bypass device in accordance with various embodiments herein.

FIG. 19 is an exploded view of a catheter connection manifold bypass device in accordance with various embodiments herein.

FIG. 20 is a schematic view of a catheter connection manifold bypass device in accordance with various embodiments herein.

FIG. 21 is a schematic view of a catheter connection manifold bypass device in accordance with various embodiments herein.

FIG. 22 is a schematic view of a catheter connection manifold bypass device in accordance with various embodiments herein.

FIG. 23 is an exploded view of a catheter connection manifold bypass device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Current systems for delivering radioactive microspheres as a form of cancer brachytherapy can include connecting an assembly that generates a suspension of microspheres in a carrier fluid with a fluid delivery catheter that is inserted into the patient receiving therapy. In specific, an outflow line of the suspension generation assembly typically connects to a connection manifold at the proximal end of the fluid delivery catheter. The suspension of microspheres passes from the generation assembly to the fluid delivery catheter and into the patient.

In order for the delivery catheter to be compatible connecting to a wide variety of devices, the catheter uses a connection manifold that conforms to an international standard (e.g. ISO 80369 for luer lock connections). However, the specific geometry of the ISO standard includes a step-change in the inner diameter forming a cavity that acts as a recirculation zone and includes regions of low fluid flow rate just inside the connection manifold. This can result in microspheres undesirably accumulating or otherwise collecting at this point and, in some cases, ultimately being retained within the catheter instead of being delivered to the patient

Reduction of microsphere retention within the catheter is important for delivering optimal therapy to the patient and enhancing usability of the system for clinicians. For example, reducing microsphere retention within the system maximizes delivery of therapeutic radiation to the patient. It is even more important in scenarios where dosages are relatively small (such as in the treatment of glioblastoma) as the starting delivery dose may be relatively small and the retention of microspheres can impact the quantity of microspheres that reach the targeted therapy site for the patient. Reducing microsphere retention also enhances therapy control and consistency of the same.

Currently, clinicians try to address microsphere accumulation issues in various ways. As one example, clinicians position the connection of the suspension generation assembly and the fluid delivery catheter in a vertical orientation with respect to gravity. This makes the recirculation zone less likely to capture microspheres because gravitational force is likely to pull microspheres out of the recirculation zone during pauses in the flushing and towards the distal end of the catheter manifold. As another example, clinicians may physically tap on the connection manifold area of the catheter during delivery to address microsphere accumulation. However, these approaches may not be convenient and/or fully effective.

Embodiments herein include a manifold bypass accessory device that can enable more efficient expulsion and delivery of microspheres to the patient by preventing microsphere accumulation in the connection manifold of the delivery catheter. Specifically, the manifold bypass accessory devices herein can streamline the flow path of microsphere suspension and allow the microspheres to effectively bypass some or all of the recirculation zone making it unlikely that microspheres will be captured by the same. Devices herein can also enable the efficient delivery of the microspheres without taking measures such as orienting the catheter connection manifold in a vertical position (e.g., the connection manifold can be oriented in any manner desired including a horizontal orientation when using embodiments herein) and without requiring other manipulation (such as tapping of the connection manifold). In addition, the devices herein can increase delivery/microsphere clearance within the system, reduce the amount of flushing needed in the procedure, and reduce the amount of time required per procedure.

Beneficially, devices herein can integrate into existing delivery systems and can be used across various microcatheters regardless of connection manifold geometry. Manifold bypass devices herein can be used with microcatheter and tubing sets of various sizes. For example, devices herein can be used with microcatheter inner diameters ranging from 0.010″ (or 0.254 mm) (such as may be used with glioblastoma) to 0.028″ (or 0.7112 mm) (such as may be used with hepatocarcinoma) and suspension generation assembly tubing inner diameters that are from 0.020″ (0.508 mm) to 0.040″ (1.016 mm), or even smaller or larger. In various embodiments, the inner diameter of the bypass device can be selected to be between the interior diameters of the outlet tubing and the microcatheter.

In an embodiment, a catheter connection manifold bypass device can be included having a fluid passage conduit with a proximal end and a distal end. The bypass device can also include a deformable tip, wherein the deformable tip can be disposed around the distal end of the fluid passage conduit. The deformable tip can be configured to fit within a distal portion of a cavity within a connection manifold for a fluid delivery catheter. The bypass device can include a proximal connection port that can be connected to the proximal end of the fluid passage conduit. The bypass device can also include a connection adapter defining a channel, wherein the fluid passage conduit passes through the channel. The distal end of the connection adapter can be configured to attach to a proximal end of the fluid delivery catheter.

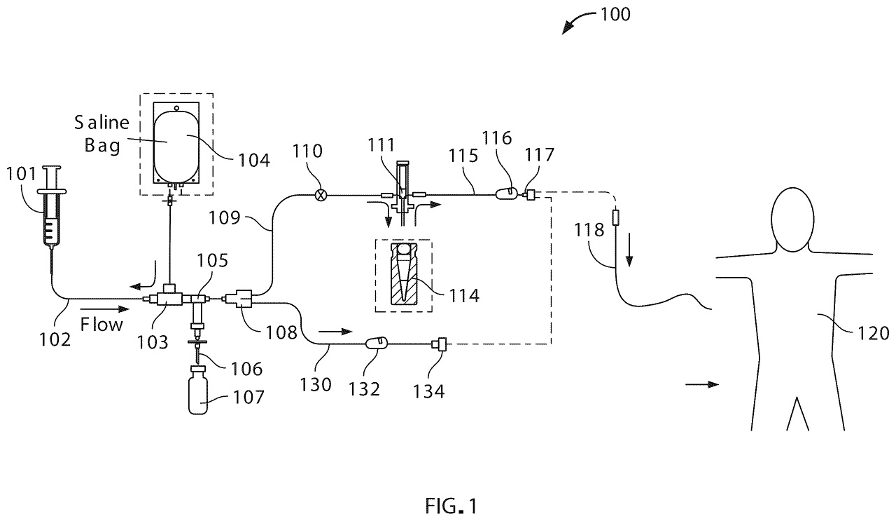

Referring now to FIG. 1, a schematic diagram is shown of components of an exemplary cancer-therapy delivery system 100 in accordance with various embodiments herein. Major parts of the cancer therapy delivery system 100 include a therapeutic fluid delivery device 101 (which in some cases can take the form of a syringe or syringe-like device), a fluid supply tube or line 102, and a dual check valve 103. In this example, the cancer-therapy delivery system 100 also includes a saline supply reservoir 104 (or fluid reservoir). The saline can serve as a carrier fluid to be mixed with the microspheres. The saline solution can be at various concentrations such as (0.3%, 0.5%, 0.7%, 0.9%, or the like). In some embodiments, the carrier fluid (typically a saline solution) can also include one or more other components. For example, in some embodiments the carrier fluid can include heparin (in the case of saline, a heparinized saline solution).

The system 100 can also include pressure relief valve 105, vented spike 106, overflow vial 107, “Y” fitting 108, fluid line 109, and check valve 110. The cancer-therapy delivery system 100 can also include a fluid injector and withdrawal assembly 111 along with a radioactive microsphere supply reservoir/mixing chamber assembly 114. The system 100 can also include outflow line 115, pinch clamp 116 and outflow connector 117.

The cancer-therapy delivery system 100 can also include and/or be connected to a fluid delivery catheter 118. A manifold bypass accessory device (described below) can be disposed between the suspension generation assembly (and specifically the outflow line 115 and/or outflow connector 117 thereof) and the fluid delivery catheter 118.

FIG. 1 also shows a patient 120 into which the fluid delivery catheter 118 can be inserted to deliver the therapeutic suspension of microspheres. In some embodiments, the fluid delivery catheter 118 can specifically be one with a relatively small diameter, such as a microcatheter with a neuro use indication (hereafter “neurocatheter”). In some embodiments, the fluid delivery catheter 118 diameter can be as small as 0.33 millimeters (mm) (0.013 inches), or even less. However, in other embodiments the catheter or microcatheter can be larger in diameter. In some embodiments, the catheter or microcatheter can have a diameter of less than or equal to 3.00, 2.67, 2.34, 2.01, 1.68, 1.35, 0.99, 0.66, 0.33, or even 0.254 mm (0.118, 0.105, 0.092, 0.079, 0.066, 0.053, 0.039, 0.026, 0.013, or even 0.010 inches, or a diameter falling within a range between any of the foregoing.

While not intending to be bound by theory, tubing and microcatheter inner diameters greater than a certain amount can lead to undesirable microsphere dropout. As such, in various embodiments herein, the inner diameter of the microcatheter (or the inner diameter of a fluid passage within the catheter) can be quite small. For example, in some embodiments, the fluid delivery catheter 118 inner diameter can be less than or equal to 0.050, 0.045, 0.040, 0.035, 0.030, 0.035, 0.020, 0.015, 0.010 inches (1.27, 1.143, 1.016, 0.889, 0.762, 0.508, 0.381, or 0.254 mm), or a size falling within a range between any of the foregoing.

In use, various operations can be performed to prepare the system 100. For example, operations can be formed such as assembly, system priming, air/bubble removal, and the like. In the context of the system configuration of FIG. 1, additional components can be utilized during such preparatory operations. By way of example, priming line 130, pinch clamp 132, and priming line connector (or luer connector) 134 can be utilized. In some preparatory operations (e.g., priming, bubble removal, etc.), the priming line 130 (and specifically the priming line connector 134) can be connected to outflow connector 117. Then, a fluid can be pulled in from saline supply reservoir 104 and then pushed through the system using the therapeutic fluid delivery device 101, including first pulling in fluid from saline supply reservoir 104, causing fluid (such as saline) to be withdrawn from the saline supply reservoir 104. Then, with fluid injector and withdrawal assembly 111 not connected to mixing chamber assembly 114, the fluid can flow through fluid delivery device 101, dual check valve 103, pressure relief valve 105, before reaching “Y” fitting 108. At that point the fluid can follow one path through check valve 110 and into fluid injector and withdrawal assembly 111. The fluid can also follow another path through priming line 130, pinch clamp 132, and priming line connector 134, entering the other side of fluid injector and withdrawal assembly 111. The fluid can then pass out of fluid injector and withdrawal assembly 111 through needles or conduits thereof described below. In this way, both sides (inflow and outflow) of the fluid injector and withdrawal assembly 111 can be primed. This can be performed until all bubbles are removed from the system.

In general, priming operations are performed before introducing the fluid injector and withdrawal assembly 111 to the dose vial to ensure that air is not introduced to the patient when starting to flush through the dose vial to the catheter. In addition, priming that includes passing fluid through the dose vial would risk moving the microspheres before priming is complete and the patient is ready to receive the microspheres. As such, after priming operations are complete, the connector 134 can be disconnected from the downstream side of fluid injector and withdrawal assembly 111. Further, the fluid injector and withdrawal assembly 111 can be connected to the mixing chamber assembly 114 and the outflow connector 117 can be connected to the fluid delivery catheter 118.

Then (omitting some possible operations for ease of explanation) the clinician or other system user can pull back on a plunger or similar mechanism of therapeutic fluid delivery device 101 causing fluid (such as a saline solution) to be withdrawn from the saline supply reservoir 104, through the dual check valve 103 and the fluid supply tube 102, and into the fluid delivery device 101. Then the clinician or other system user can depress the plunger causing fluid to flow from the therapeutic fluid delivery device 101, through the fluid supply tube 102, through the dual check valve 103, pressure relief valve 105, “Y” fitting 108, check valve 110, and into the fluid injector and withdrawal assembly 111.

The fluid injector and withdrawal assembly 111 can be in fluid communication with the mixing chamber assembly 114 and can direct a flow of fluid into the mixing chamber assembly 114 coming from the therapeutic fluid delivery device 101 or pump such as through one of a pair of needles, cannulas, or tubes serving as an inflow conduit. The fluid can become mixed with microspheres in the mixing chamber assembly 114 forming a suspension which can then exit via the fluid injector and withdrawal assembly 111 via another needle, cannula, or tube serving as an outflow conduit and through outflow line 115, pinch clamp 116, and out of outflow connector 117, though a catheter connection manifold bypass device herein, and into the fluid delivery catheter 118 and then into a desired site of the patient 120. A catheter connection manifold bypass device herein can be disposed between the outflow line 115/outflow connector 117 and the connection manifold of the fluid delivery catheter 118. The fluid delivery catheter 118 can be of various sizes. However, in some embodiments, the fluid delivery catheter 118 has a fluid delivery channel with an inner diameter of less than or equal to 0.040, 0.030, or 0.020 inches (1.016 mm, 0.762 mm, 0.508 mm). In various embodiments, the fluid delivery catheter 118 can be a microcatheter. In various embodiments, the fluid delivery catheter 118 can specifically be a neurocatheter.

After an initial volume of fluid is passed through to the patient this way, one or more flushes can be performed (e.g., additional amounts of carrier fluid can be run through the system and to the patient to ensure that all or nearly all of the intended amount of microspheres are delivered to the patient).

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. As used herein, a “lumen” or “channel” or “bore” or “passage” within a conduit or line is not limited to a circular cross-section.

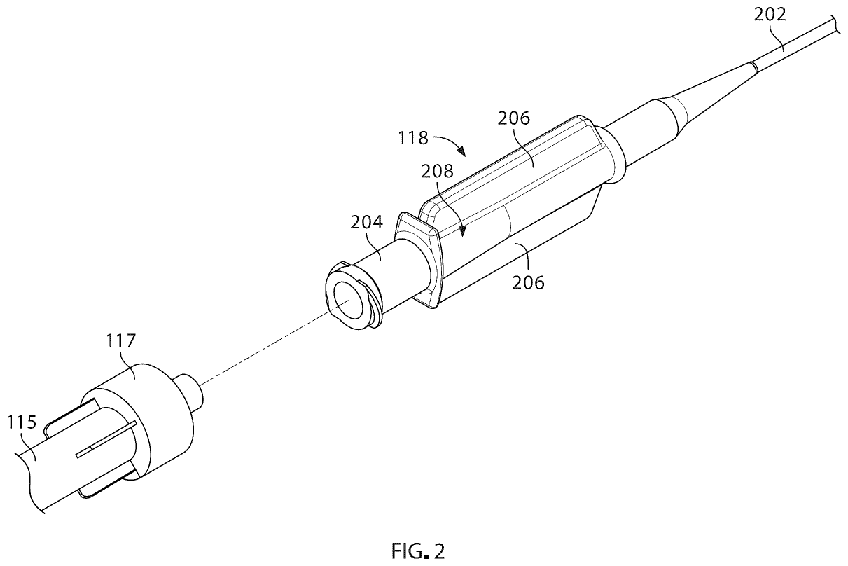

Referring now to FIG. 2, a schematic view is shown of an outflow line 115 of a suspension forming assembly along with a fluid delivery catheter 118 in accordance with various embodiments herein. The fluid delivery catheter 118 includes a catheter shaft 202 and a connection manifold 204. In this embodiment, the fluid delivery catheter 118 also includes wings 206 to aid in user manipulation of the connection manifold 204, however some catheters may omit some elements and/or include other elements. Significantly, the fluid delivery catheter 118 also includes a cavity 208 within the connection manifold 204. This cavity 208 can be much larger in diameter than the inner diameter of the catheter shaft 202 itself. In some cases, the cavity 208 can be tapered being generally larger at a proximal portion thereof versus the distal portion thereof. However, it will be appreciated that the specific geometry of the cavity 208 can vary depending on the manufacturer of the fluid delivery catheter 118. Unfortunately, the cavity 208 can serve as a recirculation zone along the fluid flow path of therapeutic suspensions generated herein and can serve to collect therapeutic microspheres otherwise delivered as a part of cancer therapy. This can lead to undesirable accumulation or retention of therapeutic microspheres preventing the same from being delivered as part of a planned therapeutic dose.

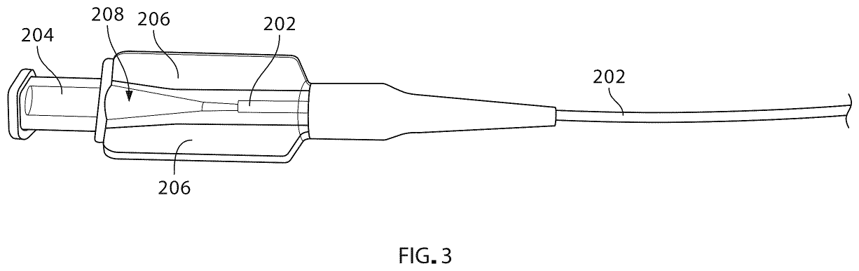

Referring now to FIG. 3, a schematic view of a portion of a fluid delivery catheter 118 is shown in accordance with various embodiments herein. As before, the fluid delivery catheter 118 includes a catheter shaft 202 and a connection manifold 204 along with wings 206. FIG. 3 illustrates a cavity 208 within the connection manifold 204. As can be seen, the size of the cavity 208 can be substantial relative to the size of the catheter shaft 202 itself.

Embodiments herein can be used to bypass the recirculation zone of the cavity within the connection manifold. For example, embodiments herein can inserted into the connection manifold and extend at least partially through the cavity within the connection manifold. This can prevent microspheres from being retained within the cavity and allow for more efficient and complete delivery of the planned therapeutic dose.

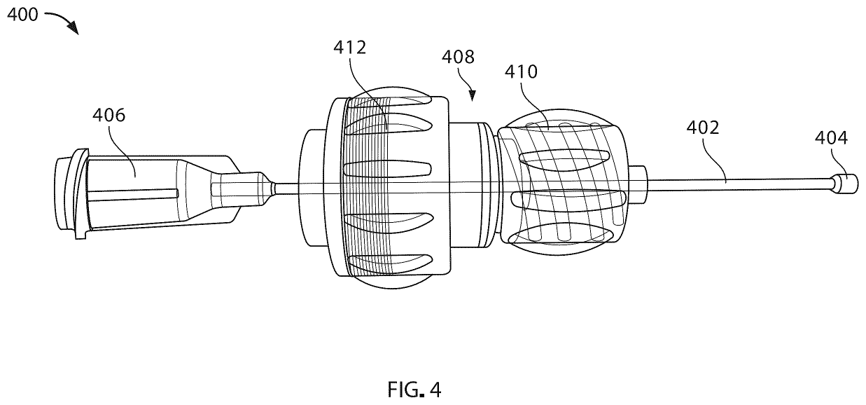

Referring now to FIG. 4, a schematic view of a catheter connection manifold bypass device 400 is shown in accordance with various embodiments herein. The catheter connection manifold bypass device 400 can be effective to reduce and/or eliminate significant step-changes in an inner diameter of a fluid passage way passing through the catheter connection manifold bypass device 400 and a fluid delivery catheter 118 when the two are connected. The bypass device 400 can allow microspheres to bypass the volume of the cavity within the catheter and provide a more streamlined flow path between the suspension generation assembly and the fluid delivery catheter lumen.

The catheter connection manifold bypass device 400 includes a fluid passage conduit 402. In various embodiments, the fluid passage conduit 402 can be configured to be advanced into a fluid delivery catheter 118 such that a distal end of a fluid passage conduit 402 comes to rest at a distal end of a cavity within a connection manifold 204 of the fluid delivery catheter 118. In various embodiments, the fluid passage conduit 402 can be formed of a metal or a polymer. In some embodiments, the fluid passage conduit 402 can take the form of a tube, hollow cylinder, duct, line, or needle or a portion thereof. In some embodiments, the fluid passage conduit 402 can be substantially rigid.

The catheter connection manifold bypass device 400 of this embodiment also includes a deformable tip 404, though not all embodiments herein may include one. The deformable tip 404 can be disposed around the distal end of the fluid passage conduit 402. In various embodiments, the deformable tip 404 can be formed of a material different than a fluid passage conduit 402. In various embodiments, the deformable tip 404 can be configured to fit within a distal portion of a cavity within a connection manifold 204 for a fluid delivery catheter 118. In various embodiments, the deformable tip 404 can be formed of an elastomeric material. Exemplary elastomers can include, but are not limited to silicone, polyurethane, ethylene-propylene rubbers (EPR), ethylene-propylene-diene rubbers (EPDM), and the like. In some embodiments, the deformable tip 404 can have an outer diameter (in a non-deformed state) that is at least 1.1, 1.2, 1.3, 1.4, 1.5, 1.75, 2, 2.5, 3, or 4 or more times the outer diameter of the fluid passage conduit 402, or an amount falling within a range between any of the foregoing. In various embodiments, the deformable tip 404 includes a tapered distal end, a hemispherical distal end, a conical distal end, or the like. Being generally deformable, the deformable tip 404 is less rigid than the fluid passage conduit 402.

The catheter connection manifold bypass device 400 also includes a proximal connection port 406. In various embodiments, the proximal connection port 406 can be configured to engage with an outflow line 115 and/or outflow connector 117 of a cancer therapy suspension forming assembly. In various embodiments, the proximal connection port 406 can be connected to a proximal end of a fluid passage conduit 402. Thus, the proximal connection port 406 can provide a linkage between the cancer therapy suspension forming assembly and the fluid passage conduit 402. In various embodiments, the proximal connection port 406 can be physically integrated with an outflow line 115 of a cancer therapy suspension forming assembly, such that a distinct outflow connector 117 and separate proximal connection port 406 is not needed. In some embodiments, the proximal connection port 406 can be threaded. In various embodiments, the proximal connection port 406 can include a luer connector. In other embodiments the proximal connection port 406 can configured to include a snap-fit or pressure-fit mechanism or can be configured to connect in other ways.

The catheter connection manifold bypass device 400 can, in some embodiments, also include a connection adapter 408. The connection adapter 408 includes an adapter distal end 410 and an adapter proximal end 412. In various embodiments, the adapter distal end 410 of the connection adapter 408 can be configured to attach to a proximal end of a fluid delivery catheter 118. In some embodiments, the adapter distal end 410 can be threaded. In various embodiments, the adapter distal end 410 can include a luer connector. In other embodiments the adapter distal end 410 can configured to include a snap-fit or pressure-fit mechanism or can be configured to connect in other ways. In various embodiments, the connection adapter 408 can be a Tuohy Borst adapter and/or function similar thereto.

The connection adapter 408 can allow the fluid passage conduit 402 to be advanced to the point of contact within the cavity of the fluid delivery catheter connection manifold. A mechanism on the connection adapter 408 (such as a threaded mechanism) can then be locked into place when a sufficient seal is obtained. For example, the connection adapter 408 can be tightened down over the outside diameter of the fluid passage conduit 402 (as a Tuohy can be tightened down) and then the adapter distal end 410 can be locked onto the microcatheter connection manifold so that no slippage occurs once a seal is made.

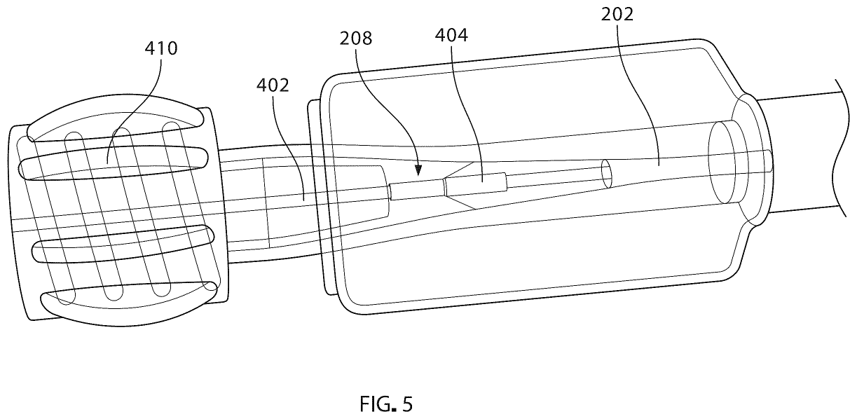

The catheter connection manifold bypass device can be inserted into the connection manifold of the fluid delivery catheter. This is illustrated with reference to FIG. 5, which is a schematic view of a catheter connection manifold bypass device 400 connected with a fluid delivery catheter 118 in accordance with various embodiments herein. As before, the fluid delivery catheter includes a catheter shaft 202. FIG. 5 also shows a cavity 208 within the connection manifold 204. The catheter connection manifold bypass device includes a fluid passage conduit 402, which can be seen passing through at least a portion of the cavity 208. The catheter connection manifold bypass device also includes a deformable tip 404. The connection adapter 408 includes an adapter distal end 410 which can be seen engaging the connection manifold 204 of the fluid delivery catheter 118. The deformable tip 404 can provide a seal with a distal portion of the interior walls of the cavity 208 or beyond the distal portion of the cavity 208 such that a suspension of microspheres herein does not flow backward around the end of the fluid passage conduit 402 and back into the cavity 208.

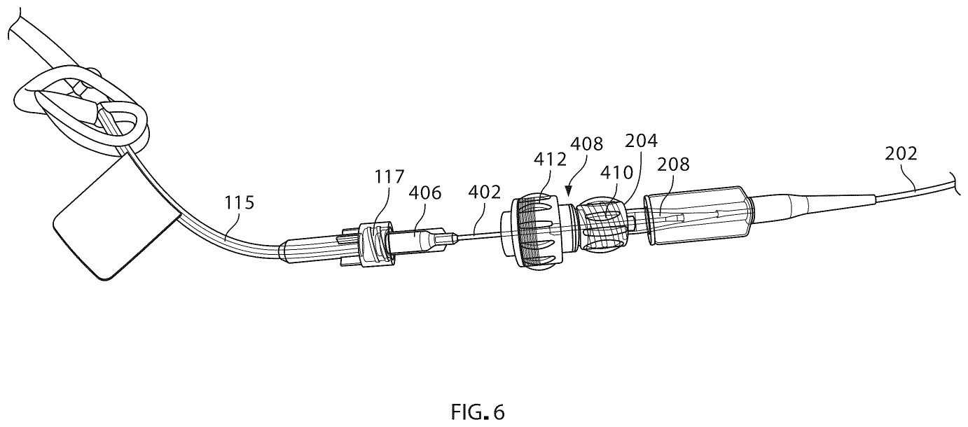

Referring now to FIG. 6, a schematic view of a portion of a suspension forming assembly and a bypass device herein is shown connecting with a fluid delivery catheter 118 in accordance with various embodiments herein. FIG. 6 specifically shows an outflow line 115 of the suspension forming assembly. As before, the fluid delivery catheter includes a catheter shaft 202 which defines a lumen therein for the passage of the suspension of microspheres. The fluid delivery catheter also includes a connection manifold 204 with a cavity therein. A catheter connection manifold bypass device includes a proximal connection port 406 which is connected to and in fluid communication with an outflow line 115 of the cancer therapy suspension forming assembly. The catheter connection manifold bypass device also includes a connection adapter 408 which includes an adapter distal end 410 and an adapter proximal end 412.

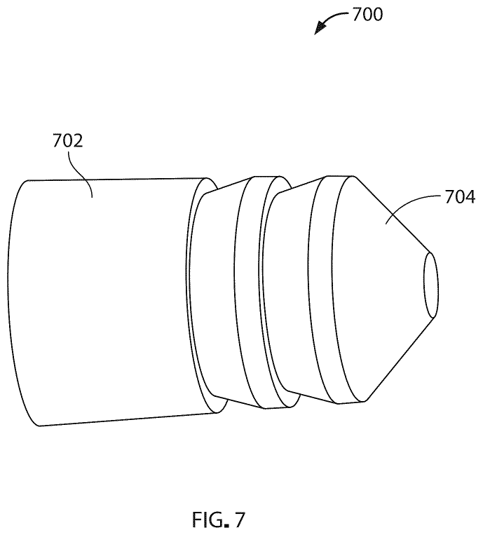

Other components can be included with some embodiments herein. By way of example, referring now to FIG. 7, a schematic view of a proximal connection port insert 700 (or needle manifold insert) is shown in accordance with various embodiments herein. The proximal connection port insert 700 includes an insert body 702. The connection port insert 700 also includes a tapered insert tip 704. The proximal connection port insert 700 can be compliant and can be inserted into proximal connection port 406 and conform to the interior walls thereof. In some embodiments it can include a funneled lumen reducing the inner diameter from the outflow line 115 to the inner diameter of the fluid passage conduit 402 (or needle) and distal ribs that latch the insert into place. When the outflow line 115 is attached onto the proximal connection port 406, the male end of the outflow line 115 compresses the insert, seating it and sealing off the manifold recirculation zone. The lumens of the outflow line 115 and the insert 700 can be aligned and the flow path can be directed into the lumen of the fluid passage conduit 402. This insert 700 can be universal across all fluid passage conduit (or needle) gauges.

The deformable tip disposed over the distal end of the fluid passage conduit provides a seal within the fluid delivery catheter connection manifold. The deformable tip has enough size and compliance to conform to whatever shape the connection manifold is. The closer the fluid passage conduit (or needle) can get to the distal end of the cavity (or even beyond it) within the connection manifold of the fluid delivery catheter the more recirculation zone is taken away by the device. Multiple different deformable tip sizes and shapes can be used and optimized so long as there is an adequate seal within the connection manifold.

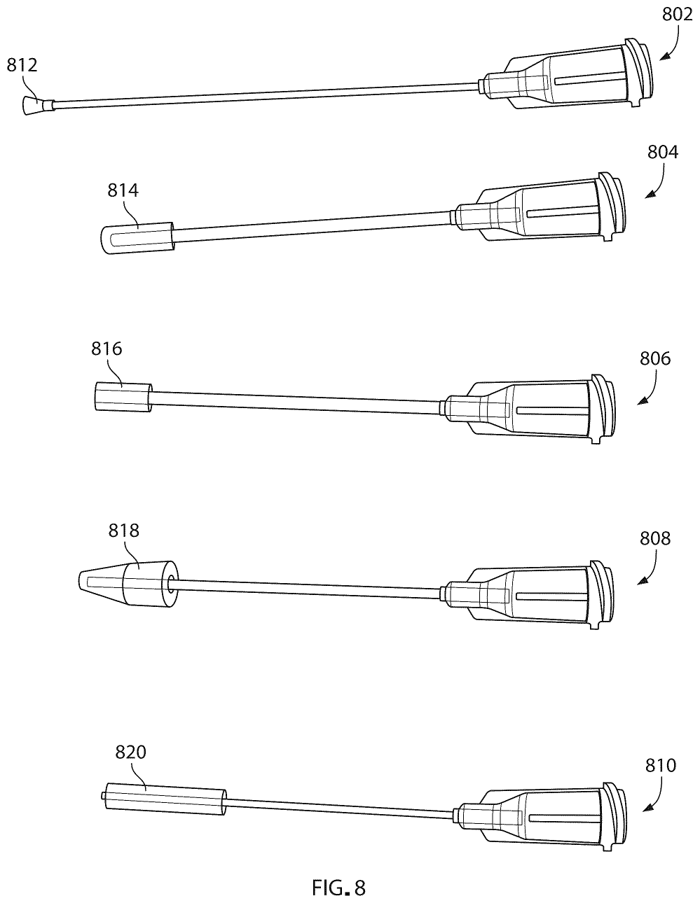

Referring now to FIG. 8, a schematic view of alternative embodiments of a fluid passage conduits and deformable tips thereof are shown in accordance with various embodiments herein. The first fluid passage conduit 802 includes a first deformable tip 812. The second fluid passage conduit 804 includes a second deformable tip 814. The third fluid passage conduit 806 includes a third deformable tip 816. The fourth fluid passage conduit 808 includes a fourth deformable tip 818. The fifth fluid passage conduit 810 includes a fifth deformable tip 820. The deformable tips each have a slightly different geometry and serve as non-limiting examples of the different forms the deformable tip can assume.

The gauge of the fluid passage conduit (or needle) can be based on outlet tubing inner diameter and microcatheter selected. In various embodiments, the inner diameter of the fluid passage conduit will be somewhere in between the outlet tubing inner diameter and the microcatheter inner diameter.

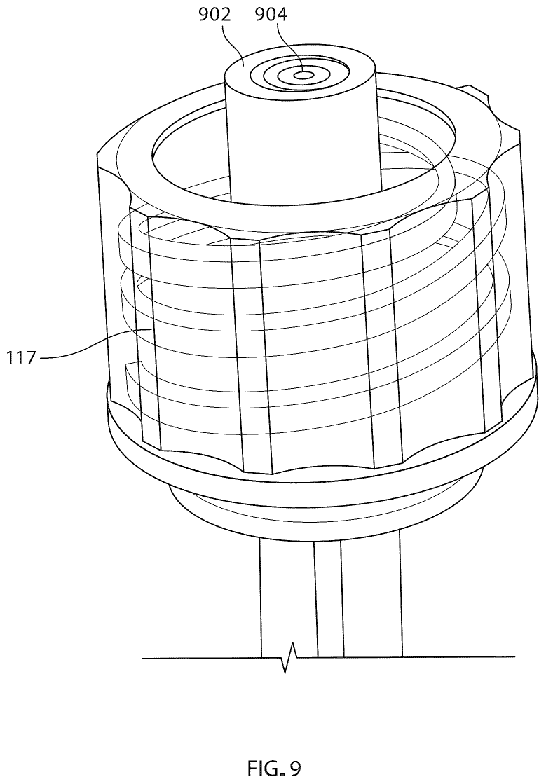

In some embodiments, a distal portion of the through outflow line 115 and/or outflow connector 117 can be modified to enhance performance herein. For example, referring now to FIG. 9, a schematic view of a distal portion of the outflow line 115 of a suspension forming assembly is shown in accordance with various embodiments herein. The suspension forming assembly of this embodiment includes a connector distal end 902 and an outflow line distal end 904. In other embodiments, the outflow line distal end 904 ends prior to the connector distal end 902. However, the transition between the outflow line distal end 904 and the connector distal end 902 can serve as a potential area to entrap microspheres. By moving the outflow line distal end 904 to align with the connector distal end 902, the potential for microsphere accumulation is minimized in that transition zone.

It will be appreciated that connection manifold bypass devices herein can also take other forms. For example, in various embodiments, a catheter connection manifold bypass device can include a fluid passage conduit with a proximal end and a distal end, along with a deformable tip, wherein the deformable tip can be disposed around the distal end of the fluid passage conduit. The deformable tip can be configured to fit within a distal portion of a cavity within a connection manifold for a fluid delivery catheter. The bypass device can also include a connection housing which can include a proximal connection fitting that can be configured to engage with an outflow line and/or distinct outflow connector of a cancer therapy suspension forming assembly. The fluid passage conduit passes through the connection housing. The manifold bypass device can also include a rotating connector element defining a channel, wherein the fluid passage conduit passes through the channel. A distal end of the rotating connector element can be configured to attach to a proximal end of the fluid delivery catheter. The rotating connector element engages with the connection housing. Rotation of the rotating connector element can cause the fluid passage conduit to advance within the connection manifold of the fluid delivery catheter and can be manipulated by a user to position the fluid passage conduit as desired.

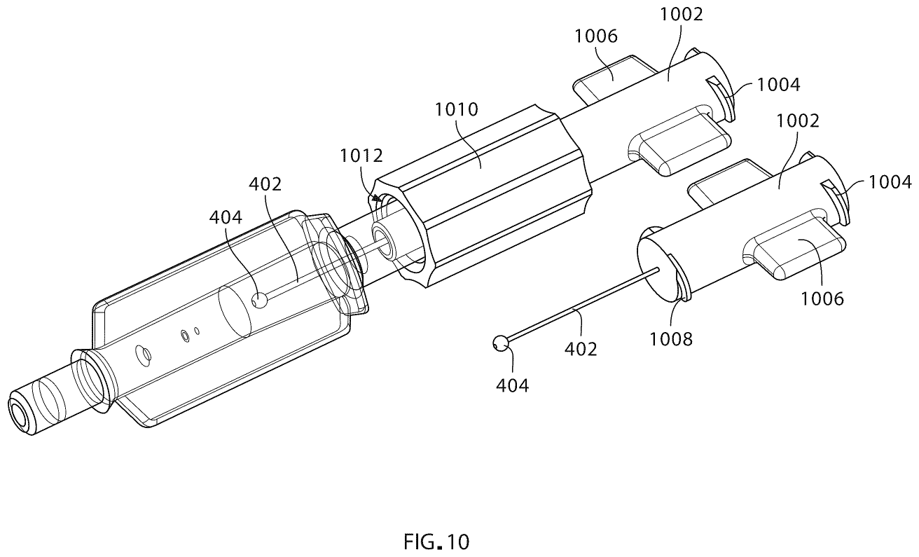

An example of this configuration is illustrated with reference to FIG. 10. FIG. 10 shows a schematic view of components of a connection manifold 204 bypass device in accordance with various embodiments herein. As before, the catheter connection manifold bypass device includes a fluid passage conduit 402 along with a deformable tip 404. However, in this embodiment, the catheter connection manifold bypass device also includes a connection housing 1002. In various embodiments, the fluid passage conduit 402 passes through the connection housing 1002.

The connection housing 1002 can include a proximal connection fitting 1004 and, optionally, grip wings 1006. In this embodiment, the connection housing 1002 also includes distal end threading 1008 and a rotating connector element 1010. The rotating connector element 1010 includes internal threading 1012.

The proximal connection fitting 1004 can be configured to engage with an outflow line 115 of a cancer therapy suspension forming assembly. In some embodiments, the proximal connection fitting 1004 can be physically integrated with an outflow line 115 and/or outflow connector 117 of a cancer therapy suspension forming assembly.

In various embodiments, a distal end of the rotating connector element 1010 is configured to attach to a proximal end of a fluid delivery catheter 118, such as the connection manifold thereof. Further, the rotating connector element 1010 can engage with the connection housing 1002. In operation, rotation of the rotating connector element 1010 can cause a fluid passage conduit 402 and the deformable tip 404 thereon to be advanced into the connection manifold of the fluid delivery catheter 118. Once a desired position is achieved, the pitch of the threading of the rotating connector element 1010 is such that the fluid passage conduit 402 is locked into place relative to the connection manifold of the fluid delivery catheter and the position is held.

In another embodiment, an internal spring-loading mechanism can be included to exert constant forward force on the fluid passage conduit 402 (and the seal between the deformable tip 404 and the interior surface of the connection manifold) and simultaneously create a sealed bridge through the fluid passage conduit 402. That way the user never loses the seal while handling the assembly. The spring-loading mechanism can use an internal spring or a compliant (such as an elastomeric) material to exert the forward force. This embodiment can be useful to accommodate a range of manifold geometries/lengths and reduce force variability and guesswork during the assembly stage of system use.

An example of such an embodiment is illustrated with respect to FIG. 11, which shows a schematic view of components of a connection manifold bypass device in accordance with various embodiments herein. As before, the catheter connection manifold bypass device includes a connection housing 1002 with a proximal connection fitting 1004 and grip wings 1006. The connection housing 1002 also includes distal end threading 1008. A rotating connector element 1010 is included having internal threading 1012. The rotating connector element 1010 also includes an insert 1102 which can be formed of an elastomeric material and which can serve as the spring-force mechanism described above to exert a forward force on the fluid passage conduit 402.

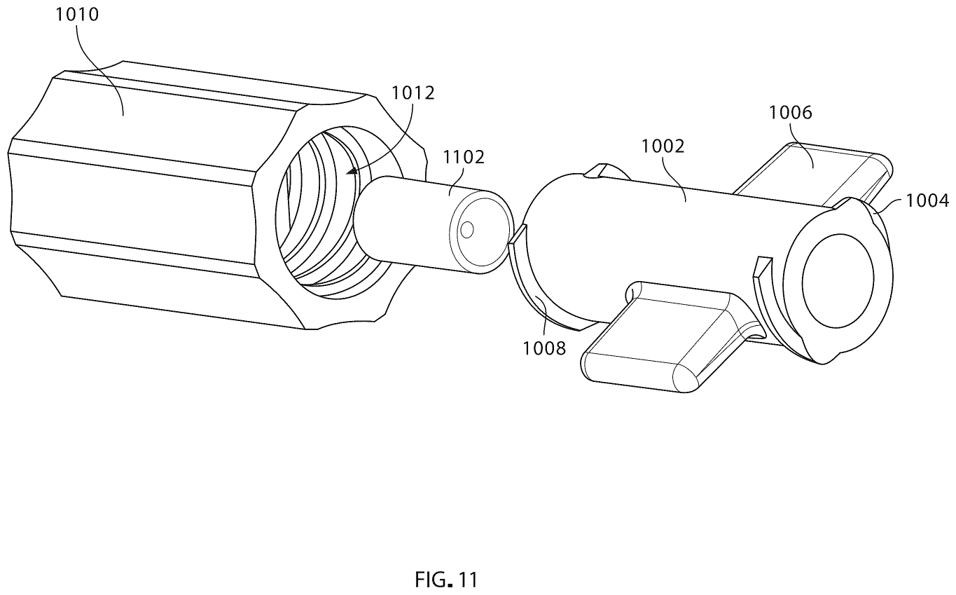

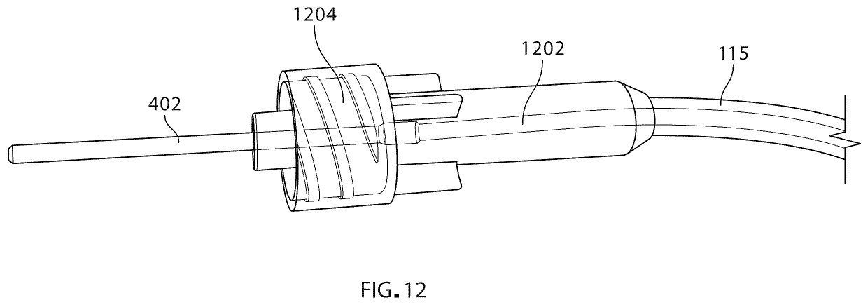

It will be appreciated that some components described herein can be physically integrated with one another. For example, the proximal connection port of other embodiments herein can be physically integrated with an outflow line of a cancer therapy suspension forming assembly. Such a design can reduce the number of components involved. Effectively, it can include a fluid passage conduit 402 directly inserted into the outlet tubing of the suspension forming assembly. This can eliminate a connector on the outlet tubing and a needle manifold insert as compared with other embodiments herein. Referring now to FIG. 12, a schematic view of a bypass device integrated with the outflow line 115 of a suspension forming assembly is shown in accordance with various embodiments herein. The cancer therapy suspension forming assembly includes an integrated connector 1202, a distal connector 1204, and a fluid passage conduit 402.

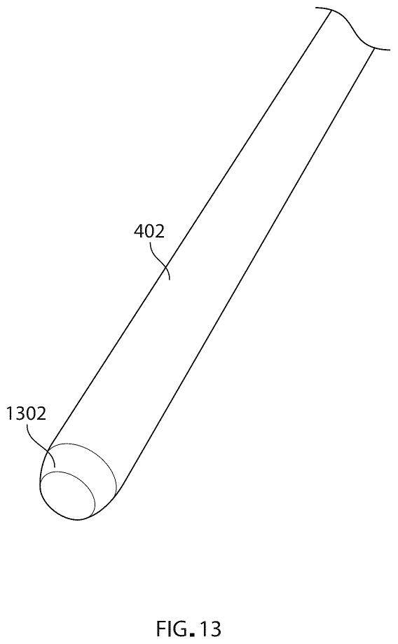

The distal end of the fluid passage conduit can have various configurations. In some embodiments, the distal end of the fluid passage conduit can be tapered to promote an efficient seal. Referring now to FIG. 13, a schematic view of a distal tip of a fluid passage conduit 402 is shown in accordance with various embodiments herein. In this embodiment, the fluid passage conduit 402 includes a tapered tip 1302 and/or a rounded tip.

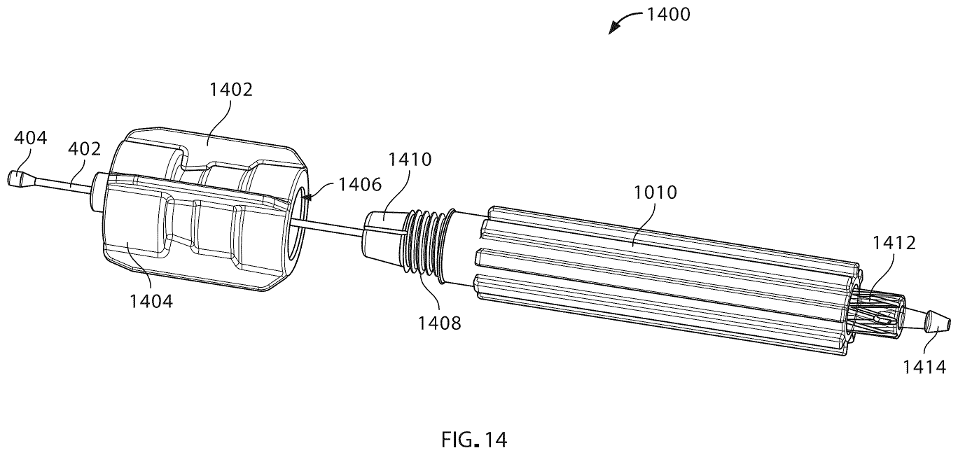

Referring now to FIG. 14, a schematic view is shown of a catheter connection manifold bypass device 1400 in accordance with various embodiments herein. In specific, FIG. 14 shows fluid passage conduit 402, deformable tip 404, and rotating connector element 1010. FIG. 14 also shows a distal connection member 1402 that includes catheter connector 1404 and adjustable insertion length receiving lumen 1406. The catheter connector 1404 can facilitate connection with the connection manifold of the catheter. In this example, the rotating connector element 1010 includes threading 1408 and locking mechanism 1410. The locking mechanism 1410 can include slots like a collet and function similarly such that when it is inserted into the receiving lumen 1406 a force is applied to the outside of the locking mechanism (such as by being pushed against the inner walls of the receiving lumen 1406) then the inner diameter of the channel (typically cylindrical) within the locking mechanism is reduced allowing it to engage with and lock the position of the fluid passage conduit 402. The threading 1408 can engage with complementary threading inside the receiving lumen 1406 such that rotating the rotating connector element 1010 causes the locking mechanism 1410 to be pushed into the receiving lumen 1406 and the resulting force exerted by the inner walls of the receiving lumen 1406 on the locking mechanism 1410 causes it to lock down onto the fluid passage conduit 402. The bypass device 1400 can also include housing insert 1412 and barb fitting 1414. The barb fitting 1414 can be used to connect to the outflow line of the suspension generation assembly.

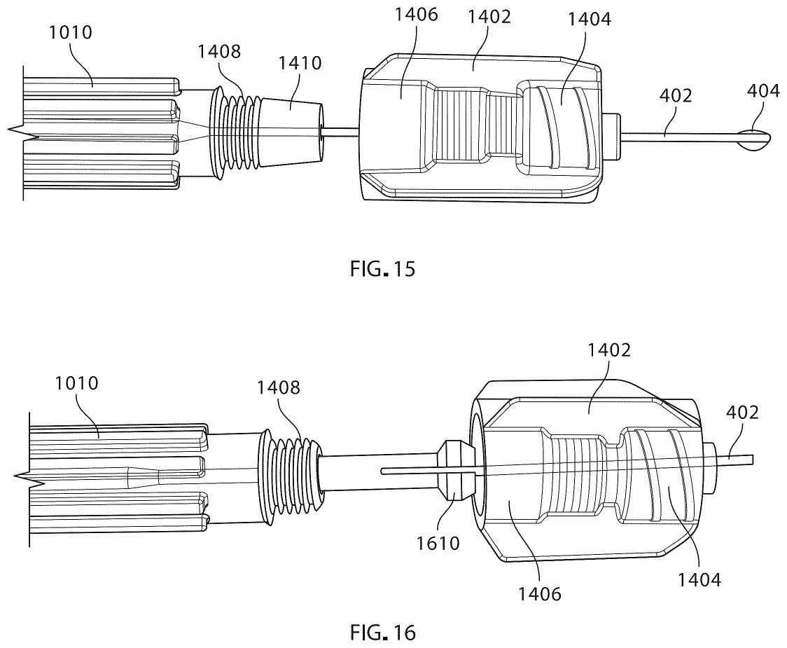

The locking mechanism 1410 can take various forms, can be integrated with other components or can be separate, and can be made of various different materials. Referring now to FIG. 15, a schematic view is shown of a catheter connection manifold bypass device in accordance with various embodiments herein. In specific, FIG. 15 shows fluid passage conduit 402, deformable tip 404, rotating connector element 1010, distal connection member 1402, catheter connector 1404, adjustable insertion length receiving lumen 1406, threading 1408, and locking mechanism 1410. In this example, the locking mechanism 1410 is physically integrated with the rotating connector element 1010.

An alternative embodiment is shown in FIG. 16 which a schematic view of a catheter connection manifold bypass device in accordance with various embodiments herein. As with FIG. 15, FIG. 16 shows fluid passage conduit 402, rotating connector element 1010, distal connection member 1402, catheter connector 1404, adjustable insertion length receiving lumen 1406, threading 1408. However, in this case a separate locking mechanism 1610 (formed of a metal) is included. However, the function of the locking mechanism 1610 can be the same or similar as that described with reference to FIGS. 14 and 15.

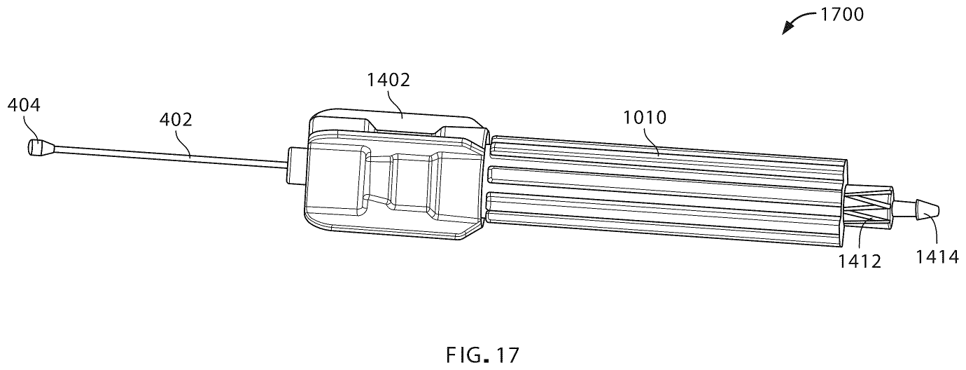

FIG. 17 is generally similar to FIGS. 14-16 and shows a schematic view of a catheter connection manifold bypass device 1700 in an assembled configuration. In specific, FIG. 17 shows fluid passage conduit 402, deformable tip 404, rotating connector element 1010, distal connection member 1402, inner housing member 1412, and connector barb 1414.

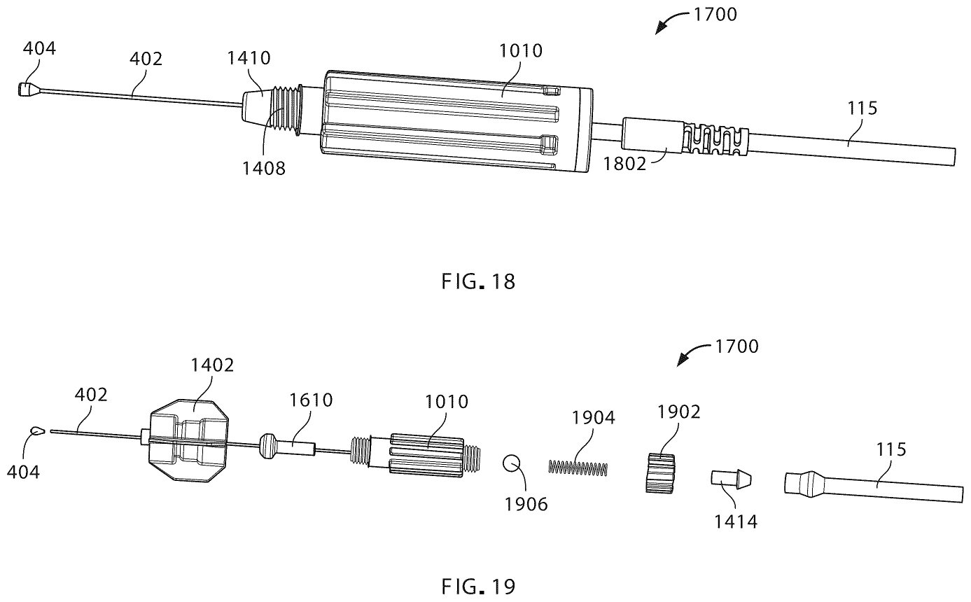

In some embodiments, a strain relief mechanism can be included to address possible issues of strain where an outflow line of a suspension generation assembly connects with a manifold bypass device herein. Referring now to FIG. 18, a schematic view is shown of a catheter connection manifold bypass device 1700 in accordance with various embodiments herein. In specific, FIG. 18 shows fluid passage conduit 402, deformable tip 404, and rotating connector element 1010. FIG. 18 also shows threading 1408, locking mechanism 1410, through outflow line 115. FIG. 18 also illustrates a strain relief mechanism 1802 which can fit over an end of the outflow line 115 where it connects with the manifold bypass device 1700.

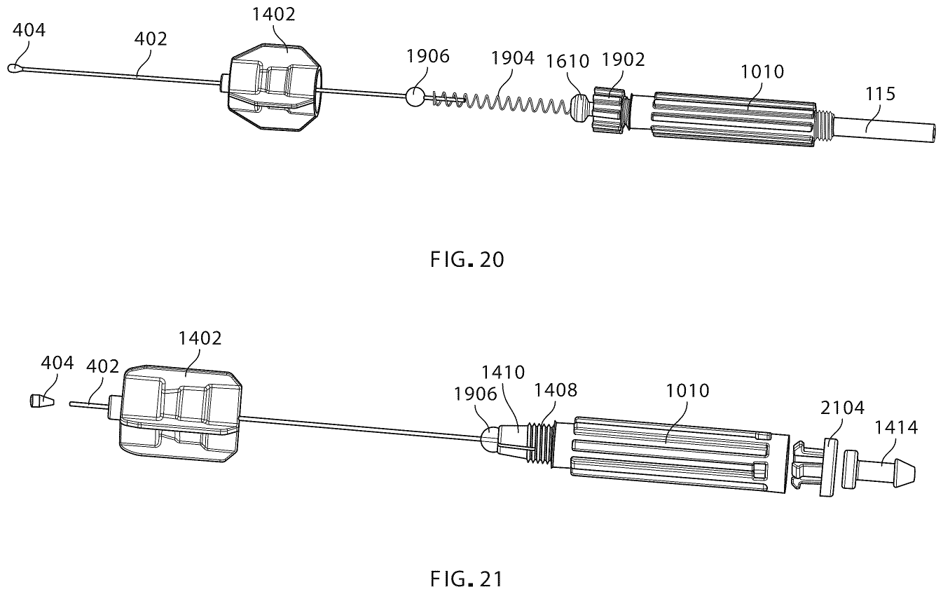

Referring now to FIG. 19, an exploded view is shown of a catheter connection manifold bypass device 1700 in accordance with various embodiments herein. In specific, FIG. 19 shows fluid passage conduit 402, deformable tip 404, rotating connector element 1010, distal connection member 1402, barb fitting 1414, through outflow line 115, and locking mechanism 1610. FIG. 19 also shows a threaded collar 1902 (which can attach to a proximal end of rotating connector element 1010 a spring 1904, and a fixated stop 1906. The spring 1904 can serve to provide a spring force on the fluid passage conduit 402 to ensure a tight seal is maintained as well as act as a limiter of the amount of force that can be applied onto the fluid passage conduit 402 in the direction of the connection with the fluid delivery catheter. The fixated stop 1906 can serve to limit the travel of the spring 1904. In some embodiments, the position of different elements herein can be rearranged. Referring now to FIG. 20, a schematic view is shown of a catheter connection manifold bypass device in accordance with various embodiments herein. In specific, FIG. 20 shows fluid passage conduit 402, deformable tip 404, rotating connector element 1010, distal connection member 1402, barb fitting 1414, outflow line 115, metal locking mechanism 1610, collar 1902, spring 1904, and fixated stop 1906.

Other configurations and components and contemplated herein. Referring now to FIG. 21, a schematic view is shown of a catheter connection manifold bypass device in accordance with various embodiments herein. In specific, FIG. 21 shows fluid passage conduit 402, deformable tip 404, rotating connector element 1010, distal connection member 1402, barb fitting 1414, outflow line 115, and fixated stop 1906. FIG. 21 also shows proximal plug 2104, which can fit onto a proximal end of rotating connection element 1010 as an alternative to threaded collar 1902.

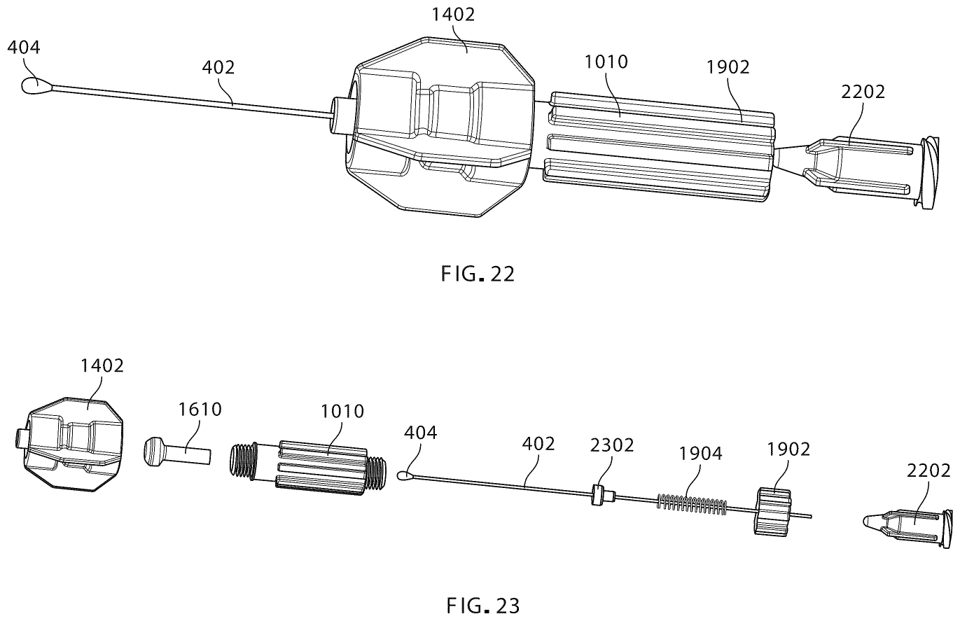

Referring now to FIG. 22, a schematic view is shown of a catheter connection manifold bypass device in accordance with various embodiments herein. In specific, FIG. 22 shows fluid passage conduit 402, deformable tip 404, rotating connector element 1010, distal connection member 1402, collar 1902. In this embodiment, a luer connection/adapter 2202 (molded or otherwise formed) is configured to be connected to a proximal end of the manifold bypass device and can interface with a distinct outflow connector 117 that is connected to through outflow line 115. Referring now to FIG. 23, an exploded view is shown of a catheter connection manifold bypass device in accordance with various embodiments herein. In specific, FIG. 23 shows fluid passage conduit 402, deformable tip 404, rotating connector element 1010, distal connection member 1402, locking mechanism 1610, collar 1902, spring 1904, luer connection/adapter 2202, and spring stop 2302.

### Microspheres

Microspheres herein can include those with a combination of yttria, alumina, and silica. By way of example, in some embodiments, microspheres herein can include Y₂O₃—Al₂O₃—SiO₂ in a 40:20:40 wt. % ratio. It will be appreciated however, that other types of microspheres are also contemplated herein.

In some embodiments, microspheres can be prepared by combining yittrium-89 with alumina and silica, in some cases also using a flame spheroidization method, and using neutron bombardment to convert Y-89 into the beta emitting radioisotope Y-90. In various embodiments, the amount of beta radiation can exceed 2500, 3000, 4000, 5000, 6000, 7000, 8000, or even 9000 Bq per sphere at the time of activity calibration (recognizing that the amount of radiation will drop after that point as the Y-90 radioisotope decays). In some embodiments, the microspheres can be provided in a vial with activity of 3 GBq or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, 0.1, 0.05, or 0.01 GBq, or less at calibration time, or an amount falling within a range between any of the foregoing.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gy, 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 20 micrometers (μm) to about 30 μm. However, in some embodiments the microspheres can be somewhat smaller or larger.

The density of the microspheres can be quite high. In some embodiments, the density of the microspheres can be above 3 g/mL, such as from 3.1 to 3.5 g/mL, or about 3.3 g/mL. By comparison, the density of water at room temperature is about 0.9978 g/mL. As such, the density of microspheres is much higher than a saline solution which influences how readily such microspheres can settle out of a suspension.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A catheter connection manifold bypass device comprising:
a fluid passage conduit, the fluid passage conduit comprising
a proximal end; and
a distal end;
a deformable tip;
wherein the deformable tip is disposed around the distal end of the fluid passage conduit;
wherein the deformable tip is configured to fit within a distal portion of a cavity within a connection manifold for a fluid delivery catheter;
a proximal connection port, wherein the proximal connection port is connected to the proximal end of the fluid passage conduit;
a connection adapter, the connection adapter defining a channel, wherein the fluid passage conduit passes through the channel; and
wherein a distal end of the connection adapter is configured to attach to a proximal end of the fluid delivery catheter.

2. The bypass device of claim 1, wherein the bypass device reduces and/or eliminates step-changes in an inner diameter of a fluid passage way passing through the bypass device and the fluid delivery catheter when the two are connected.

3. The bypass device of claim 1, wherein the fluid passage conduit is configured to be advanced into the fluid delivery catheter such that a distal end of the fluid passage conduit comes to rest at a distal end of the cavity within a connection manifold at the proximal end of the fluid delivery catheter.

4. The bypass device of claim 1, wherein the proximal connection port is configured to engage with an outflow conduit of a cancer therapy suspension forming assembly.

5. The bypass device of claim 1, wherein the proximal connection port is physically integrated with an outflow conduit of a cancer therapy suspension forming assembly.

6. The bypass device of claim 1, wherein the fluid passage conduit is rigid.

7. The bypass device of claim 1, wherein the fluid passage conduit is formed of a metal or a polymer.

8. The bypass device of claim 1, wherein the deformable tip is formed of a material different than the fluid passage conduit.

9. The bypass device of claim 1, wherein the deformable tip is formed of an elastomeric material.

10. The bypass device of claim 1, wherein the deformable tip includes a tapered distal end.

11. The bypass device of claim 1, the proximal connection port comprising a luer connector.

12. The bypass device of claim 1, wherein the connection adapter is a Tuohy Borst adapter.

13. The bypass device of claim 1, wherein the fluid delivery catheter has a fluid delivery channel with an inner diameter of less than 0.040 inches.

14. The bypass device of claim 1, wherein the fluid delivery catheter is a neurocatheter.

15. A catheter connection manifold bypass device comprising:
a fluid passage conduit, the fluid passage conduit comprising
a proximal end; and
a distal end;
a deformable tip;
wherein the deformable tip is disposed around the distal end of the fluid passage conduit;
wherein the deformable tip is configured to fit within a distal portion of a cavity within a connection manifold for a fluid delivery catheter;
a connection housing, the connection housing comprising a proximal connection fitting, wherein the proximal connection fitting is configured to engage with an outflow conduit of a cancer therapy suspension forming assembly;
wherein the fluid passage conduit passes through the connection housing;
a rotating connector element, the rotating connector element defining a channel, wherein the fluid passage conduit passes through the channel;
wherein a distal end of the rotating connector element is configured to attach to a proximal end of the fluid delivery catheter; and
wherein the rotating connector element engages with the connection housing.

16. The bypass device of claim 15, wherein the bypass device reduces and/or eliminates step-changes in an inner diameter of a fluid passage way passing through the bypass device and the fluid delivery catheter when the two are connected.

17. The bypass device of claim 15, wherein rotation of the rotating connector element causes the fluid passage conduit to be advanced into the fluid delivery catheter.

18. The bypass device of claim 15, wherein the fluid passage conduit is configured to be advanced into the fluid delivery catheter such that a distal end of the fluid passage conduit comes to rest at a distal end of the cavity within a connection manifold at the proximal end of the fluid delivery catheter.

19. The bypass device of claim 15, wherein the proximal connection fitting is physically integrated with the outflow conduit of the cancer therapy suspension forming assembly.

20. A cancer therapy delivery system comprising:
a suspension generation assembly; and
a catheter connection manifold bypass device, wherein the catheter connection manifold bypass device is in fluid communication with the suspension generation assembly, the catheter connection manifold bypass device comprising
a fluid passage conduit, the fluid passage conduit comprising
a proximal end; and
a distal end;
a deformable tip;
wherein the deformable tip is disposed around the distal end of the fluid passage conduit;
wherein the deformable tip is configured to fit within a distal portion of a cavity within a connection manifold for a fluid delivery catheter;
a proximal connection port, wherein the proximal connection port is connected to the proximal end of the fluid passage conduit;
a connection adapter, the connection adapter defining a channel, wherein the fluid passage conduit passes through the channel; and
wherein a distal end of the connection adapter is configured to attach to a proximal end of the fluid delivery catheter.
